(19) United States
(12) Patent Application Publication
(10) Pub. No.: US 2024/0358381 A1
(43) Pub. Date: Oct. 31, 2024

(54) DRILL GUIDE FOR IMPLANTABLE HEARING DEVICES

(71) Applicant: University of Rochester, Rochester, NY (US)

(72) Inventors: Anna An Guo, Rochester, NY (US); Ethan Clark Hunt, Rochester, NY (US); Samuel David Kellner, Rochester, NY (US); Abdulrahman Basim AlKhalifa, Rochester, NY (US); Anna Louise Hazelwood, Rochester, NY (US); Anne E. Luebke, Rochester, NY (US); Benjamin Thomas Crane, Rochester, NY (US)

(21) Appl. No.: 18/646,987

(22) Filed: Apr. 26, 2024

Related U.S. Application Data
(60) Provisional application No. 63/498,640, filed on Apr. 27, 2023.

(51) Int. Cl. A61B 17/17

(52) U.S. Cl. CPC A61B 17/1771

(57) ABSTRACT

In one embodiment, a drill guide for an implantable medical device includes a template comprising a turntable having a drill guide opening; and a drill clip connected to the turntable. In one embodiment, a drill guide for an implantable medical device includes a template comprising a template opening and a turntable having a drill guide opening coaxially aligned with the template opening, a drill interface connected to the turntable, and a support element connected to the drill interface extending from the turntable and offset from the drill guide opening.

### CROSS-REFERENCE TO RELATED APPLICATIONS

This application claims priority to U.S. provisional application No. 63/498,640 filed Apr. 27, 2023 incorporated herein by reference in its entirety.

### BACKGROUND OF THE INVENTION

One of the greatest problems facing patients with hearing loss (HL) is understanding speech in the presence of background noise. This issue occurs in patients with damage to their inner ear or auditory nerve (sensorineural HL), outer/middle ear (conductive HL), or a combination (mixed HL). For listeners with these types of hearing loss and single-sided deafness (SSD), cochlear implants are the gold standard for restoring binaural hearing and improving speech perception in noise, but they may not be accessible to children with agenesis of the auditory nerve and patients with destructed inner ears. As such, an alternative treatment option that requires less invasive surgery must be targeted. Bone conduction implants (BCIs) improve speech perception and have been successfully applied to SSD therapies and conductive HL patients which makes them stand as an effective alternative treatment options.

In 2018, the World Health Organization reported that 466 million people worldwide experience disabling hearing loss of up to 40 dB in adults, and 30 dB in children. Therefore, in 2018, the active transcutaneous bone conduction implant (atBCI) received FDA approval for patients 12 years and older. The most common indications for BCI candidacy include congenital aural atresia, chronic otitis externa or media, or history of obliterative reconstructive surgery. BCI devices allow for stimulation of the cochlea while bypassing malfunctional conductive aspects of the outer or middle ear.

Conductive hearing implant devices are anchored to the temporal bone and include (see for example Osia branded devices). Conductive hearing Implants are BCIs used to treat patients with either conductive or mixed hearing loss, as well as single sided deafness up to 55 dB. These devices stimulate the cochlea through bone conduction by vibrating the temporal bone, bypassing the damaged outer and/or middle ear. The device is guided by an external sound processor that is able to directly vibrate the bone through a piezoelectric transducer integrated into the mastoid bone via an osseointegrated screw (part BI300 in FIG. 1). However, there lies an important challenge during BCI implantation surgery involving the screw's angle of alignment with the mastoid bone.

Before surgery, computed tomography scans are performed to measure bone thickness and detect any abnormalities in bone structure. This is also done to determine the optimal position of implantation. The surgery begins by marking the position of the BCI components (see e.g. FIG. 1) behind the ear. The BCI is implanted behind the ear suffering hearing loss, recommended to be placed in the sinodural angle as shown in FIGS. 1B and 1C.

The skeletal muscles and the periosteum are removed as it is necessary for preventions of scar formation. A guide drill is then used alongside a widening drill to drill into a depth of 3-4 mm, depending on the bone thickness of the patient. The BCI device is then placed while a clearance indicator is placed on the guide pin of the BCI device and any protruding bone is usually removed to avoid contact of device and implant and ensure proper functionality. Then, the position of the lead exit of the actuator is marked and drilled such that the fixation interface can be visible, and a screw perpendicular to the bone surface can be placed to stabilize the device.

The inconvenience of the surgery occurs with the fixation screw where it is difficult to ensure a perfectly perpendicular alignment between the fixation interface and the patient's temporal bone. When the screw is not driven in perpendicular to the temporal bone, causing the device not to sit flat against the skull (see e.g. FIG. 1C), the coil and waist of the device protrude out unsupported. This overhang can act as a lever which puts stress on the site of attachment, as well as causing the skin to “tent” out.

To ensure effective sound transmission while avoiding loosening of the device and pain due to stretching of the skin, the implant must be perpendicular to the skull so the device lays flat. Surgeons performing BCI implantation surgery currently use a disposable template to determine the placement of the device, then rely on their experience to ensure they are drilling perpendicular to the bone. Intuition alone is not reliable, therefore a new device is needed to guide the drill and facilitate proper implantation.

Accordingly, there is a need in the art for a device that can guide drilling perpendicularity to ensure the implant fits right the first time, thus reducing correction time in the operating room, saving money and providing optimal patient outcomes. Embodiments described herein fit this need while further providing sterilizability, stability, visibility assistance, and the ability to guide perpendicular drilling at different depths.

### SUMMARY OF THE INVENTION

In one embodiment, a drill guide for an implantable medical device includes a template comprising a turntable having a drill guide opening; and a drill clip connected to the turntable. In one embodiment, the turntable is at least partially seated in a bearing connected to the template. In one embodiment, the bearing comprises a bearing opening aligned with the drill guide opening. In one embodiment, the bearing is at least partially seated in a template recess. In one embodiment, the template recess comprises a template recess opening coaxially aligned with the drill guide opening and the bearing opening. In one embodiment, the drill clip is connected to the turntable by a support element extending above the turntable. In one embodiment, the drill clip is slidably connected to the support element. In one embodiment, the support element is configured to restrict slidable drill movement perpendicular to the target site when a drill is attached to the drill clip. In one embodiment, the support element comprises a plurality of separate support elements. In one embodiment, the support element extends from an offset position above the turntable. In one embodiment, the template is an implantable hearing device template. In one embodiment, the implantable medical device is an implantable hearing device.

In one embodiment, a drill guide for an implantable medical device includes a template comprising a turntable having a drill guide opening; and a drill clip connected to the turntable and configured for slidable movement restricted to perpendicular to the drill guide opening.

In one embodiment, a drill guide for an implantable medical device includes a template comprising a turntable having a drill guide opening; and a slidable drill clip connected to the turntable above the turntable.

In one embodiment, a drill guide for an implantable medical device includes a substrate comprising a turntable configured to turn at least partially about a drill guide opening; and a drill clip configured to turn at least partially about the drill guide opening.

In one embodiment, a drill guide for an implantable medical device includes a template comprising a template opening and a turntable having a drill guide opening coaxially aligned with the template opening; and a drill interface connected to the turntable; a support element connected to the drill interface extending from the turntable and offset from the drill guide opening. In one embodiment, the support element is slidably connected to the drill interface. In one embodiment, the turntable is at least partially housed in a template recess. In one embodiment, the drill guide includes a ring bearing seated in the template recess and coaxially aligned with the drill guide opening and template opening.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is a prior art figure showing components of a BCI hearing aid device with an external audio processor; FIG. 1B is a prior art figure showing implantation of a BCI hearing aid device, FIG. 1C is a prior art figure showing (left to right) sagittal and frontal views of a BCI implant not placed flat against the skull, illustrating how the overhanding area acts as a lever which can pry the implant away from the skull, as well as causing the skin to tent, FIG. 1D is a prior art diagram showing improper and proper implantation, and FIG. 1E is a prior art diagram showing a conventional template and position for drilling and inserting the implant.

FIGS. 2A-2H are views of a drill guide according to one embodiment; FIG. 2A showing an assembled perspective view of a drill guide, FIG. 2B showing a perspective view of the drill clip in isolation, FIG. 2C showing a top-down view of a turntable in isolation, FIG. 2D showing a magnified cutaway view of the drill guide's turntable, bearing and template interface, FIG. 2E showing an exploded view of the drill guide, FIG. 2F showing a perspective view of the drill guide without the clip, FIG. 2G showing a functional drilling view, and FIG. 2H showing a functional sanitization view.

FIGS. 3A-3E show alternate embodiments; FIG. 3A showing a channel that can be used to stop depth and maintain perpendicularity, FIG. 3B showing a separating channel that can be used to stop depth and maintain perpendicularity, FIG. 3C showing a pillared channel that can be used to stop depth and maintain perpendicularity, FIG. 3D showing a 2-piece clip that can be used to limit drill movement, and FIG. 3E showing a slidable clip without the turntable.

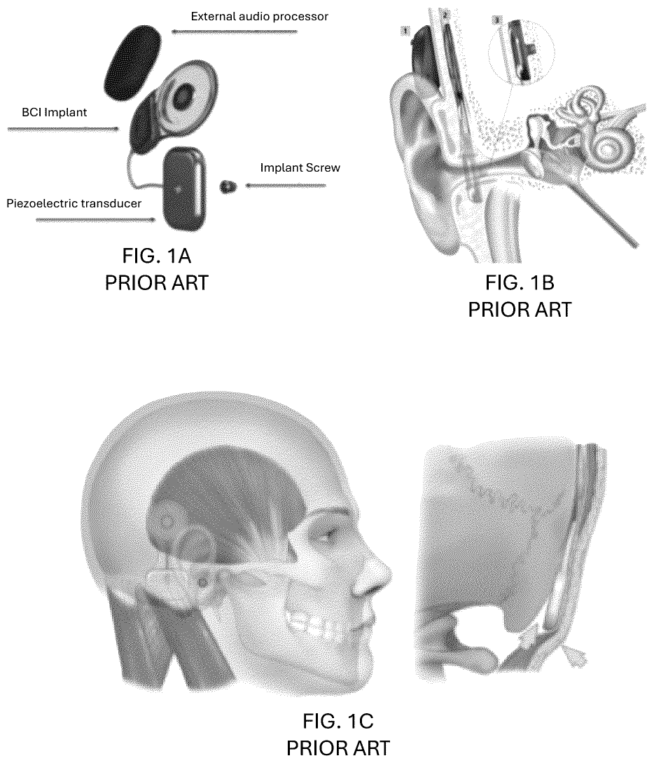

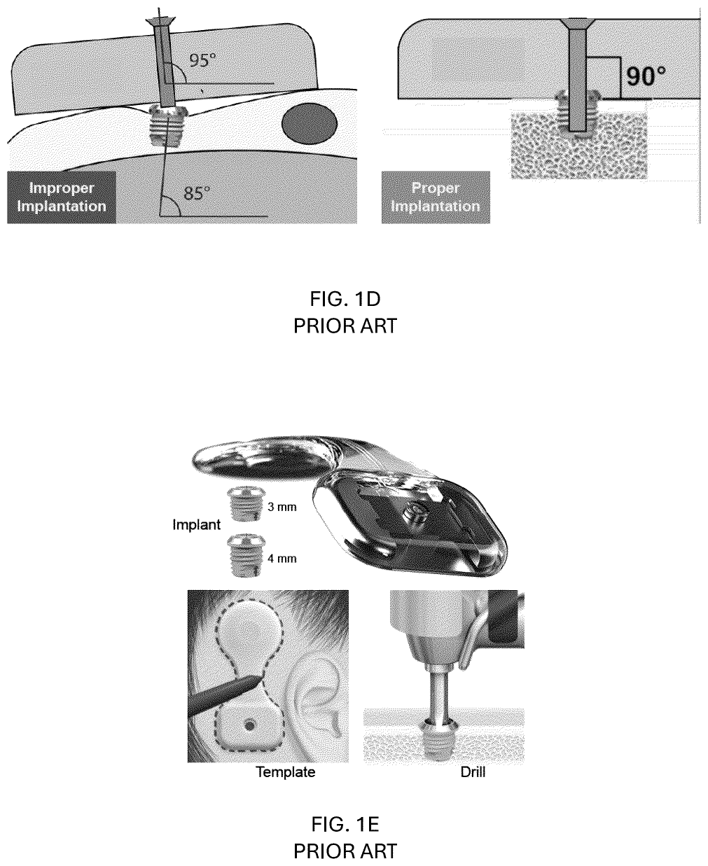

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in drill guides for implantable hearing devices. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a drill guide for implantable hearing devices.

Embodiments of the drill guide device disclosed herein are designed for improved screw alignment during implantation surgeries for medical devices such as conductive hearing implants. In certain embodiments, intended use is for maintaining a right angle between the screw and the skull surface to ensure a snug fit of the device against the skull. Intended customers for this device include otolaryngologists and residents who perform BCI implantation surgeries.

Advantages of the device may include for example reduced recovery time from surgery, reduced caretaker time, reduced time required for surgery preparation, reduced length of surgery and amount of anesthesia required, reduced cost of surgery or extra visits due to complications, and reduced strain on hospital resources. Other advantages and aspects of the embodiments include for example: alignment of the drill bit perpendicular to the surface of the skull (e.g. within 3 degrees or less); allowing the device to attach to a sit flat against the skull; surgeon ability to select multiple depths (e.g. 3 mm for children or people with thinner skulls, or 4 mm for average adults); surgeon ability to accurately drill to an intended depth (e.g. +/−0.1 mm); surgeon ability to select multiple bit diameters; device ability to bend over uneven bone surfaces (semi-rigid and flexible, e.g. utilizing a 150-500 MPa polymer); ability to sterilize to prevent infections (e.g. melting point above 132 degrees Celsius); ease of use during surgical procedures for reducing average surgical time; reduction of surgical failure rates; and a non-slip surface (e.g. utilizing a polymer having a 0.2 frictional coefficient with addition of surface features such as knurling, ridges, protrusions and recesses for improving frictional force).

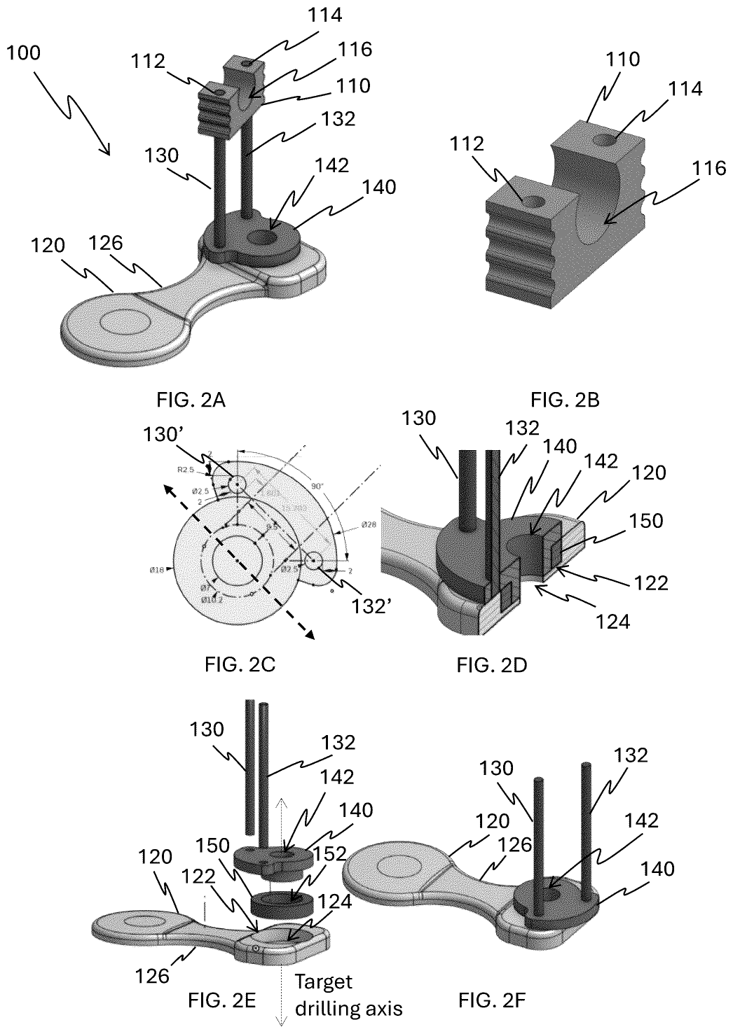

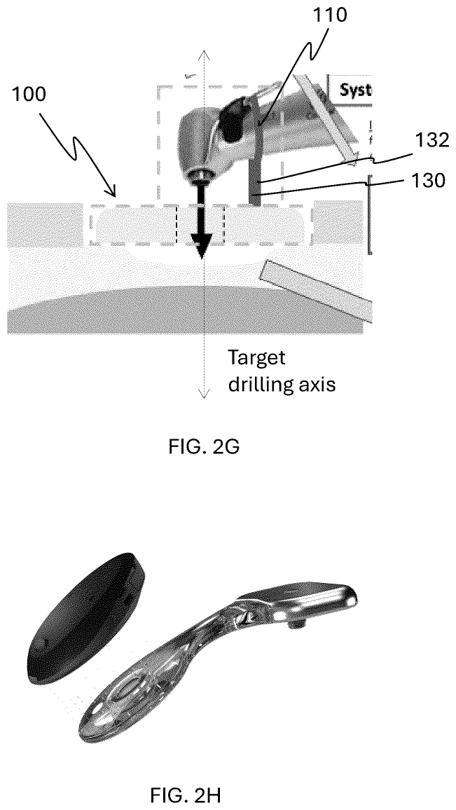

With reference now to FIGS. 2A-2H, a drill guide 100 for an implantable medical device is described according to one embodiment. The drill guide 100 has a template 120 that has flexibility to lay flat across the patient's anatomy. Template 120 flexibility may for example be maximized at the neck 126 with more rigidity and/or thickness towards the opposing ends. Surface features such as protrusions or material compositions with greater slip resistance may be utilized at skin contacting surfaces to best maintain position on the skin. Medical grade polymers such as polyvinyl chloride (PVC), polyethylene (PE), polypropylene (PP), and thermoplastic elastomers (TPEs) may be used.

A turntable 140 has a drill guide opening 142 and a clip 110 is connected to the turntable 140 for stabilizing drill position perpendicularly. The clip 110 includes a first channel 112 and second channel 114 for interfacing with support members 130 and 132 respectively. A recessed area 116 such as a U-shaped recess opens to a top side of the clip for accepting insertion of and stabilizing a drill. Multiple clips with various recessed area geometries can for example come in a kit for fitting common drill models. The clip fits snugly onto the next of the drill and provides up and down sliding movement as well as rotation around the drill hole while preventing tilting and maintaining a perpendicular approach. As a drop-in component the drill clip is each to swap out as needed. The drill clip 110 can be any securement mechanism utilizing methods known for stabilizing tool movement in a tool guide. For example, a key or hand tightening mechanism can be used to secure proper drill position on the clip. One or more clamping systems, snap-fit geometries, locking screws or bolts, and magnetic securements can also be utilized.

The turntable 140 is at least partially seated in a bearing 150 connected to the template 120. The bearing 150 can be ring shaped having a bearing opening 152 aligned with the drill guide opening 142. The bearing 150 is at least partially seated in a template recess 122. The template recess 122 has a template recess opening 124 coaxially aligned with the drill guide opening 142 and the bearing opening 152.

When the drill is properly positioned perpendicular and the bit is centered along the target drilling axis extending centrally though the openings, the drill can move up and down and rotate while mainlining drill bit position along the target drilling axis, perpendicular to the bone. The drill clip 110 is connected to the turntable 140 by a support element or as shown in the figures a pair of support elements 130, 132 extending above the turntable 140. The support elements 130, 132 slidably fitted within channels 112, 114 allow the drill clip 110 to slide up and down perpendicular to the turntable 140 and template 120. The support elements 130, 132 can be posts or rods configured to restrict slidable drill movement perpendicular to the target site when a drill is attached to the drill clip 116. The support elements extend from an offset position above the turntable 140 for maximizing surgeon visibility to the surgical site. Other types of mechanical elements known in the art can be utilized for producing perpendicular movement along the linear pathway, such as linear bearings or bushings (e.g. providing low-friction support for shafts or rods), rails, guides such as slotted, sliding or dovetail guides (e.g. two surfaces sliding against each other to produce linear movement), or mechanical linkages such as sliders, cranks, and link arms configured for linear motion.

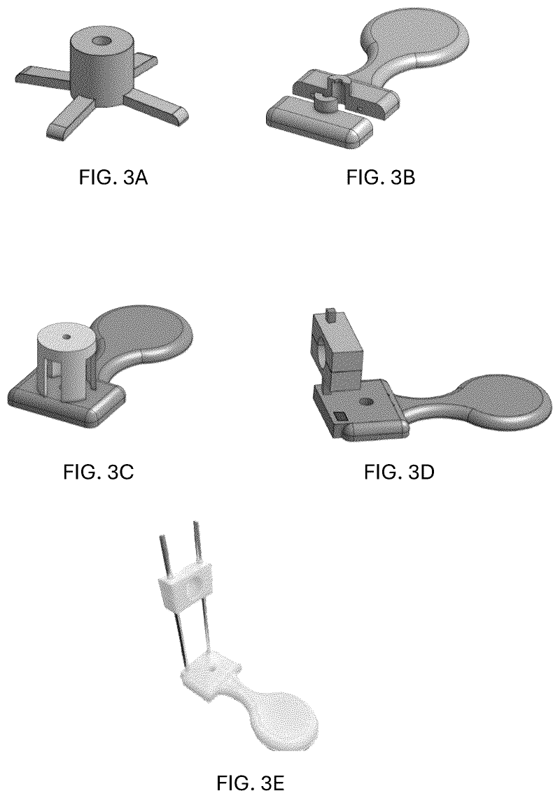

Alternate embodiments are shown in FIGS. 3A-3E. As would be apparent to those having ordinary skill in the art, features from each embodiment can replace or be combined with other embodiments described herein. For example as shown in FIGS. 3A-3C, a raised channel that can be used to stop depth and help maintain perpendicularity. The channel can be solid (FIG. 3A), separable (FIG. 3B) and/or pillared (FIG. 3C) and can be incorporated into the turntable, template or a combination of components. As shown in FIG. 3D, a 2-piece clip can be used to fit the drill in a surrounding configuration for limiting drill movement. As shown in FIG. 3E, the slidable clip can be implemented without the turntable.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A drill guide for an implantable medical device comprising:
a template comprising a turntable having a drill guide opening; and
a drill clip connected to the turntable.

2. The drill guide of claim 1, wherein the turntable is at least partially seated in a bearing connected to the template.

3. The drill guide of claim 2, wherein the bearing comprises a bearing opening aligned with the drill guide opening.

4. The drill guide of claim 2, wherein the bearing is at least partially seated in a template recess.

5. The drill guide of claim 4, wherein the template recess comprises a template recess opening coaxially aligned with the drill guide opening and the bearing opening.

6. The drill guide of claim 1, wherein the drill clip is connected to the turntable by a support element extending above the turntable.

7. The drill guide of claim 6, wherein the drill clip is slidably connected to the support element.

8. The drill guide of claim 6, wherein the support element is configured to restrict slidable drill movement perpendicular to the target site when a drill is attached to the drill clip.

9. The drill guide of claim 6, wherein the support element comprises a plurality of separate support elements.

10. The drill guide of claim 1, wherein the support element extends from an offset position above the turntable.

11. The drill guide of claim 1, wherein the template is an implantable hearing device template.

12. The drill guide of claim 1, wherein the implantable medical device is an implantable hearing device.

13. A drill guide for an implantable medical device comprising:
a template comprising a template opening and a turntable having a drill guide opening coaxially aligned with the template opening;
a drill interface connected to the turntable; and
a support element connected to the drill interface extending from the turntable and offset from the drill guide opening.

14. The drill guide of claim 13, wherein the support element is slidably connected to the drill interface.

15. The drill guide of claim 13, wherein the turntable is at least partially housed in a template recess.

16. The drill guide of claim 15 further comprising:
a ring bearing seated in the template recess and coaxially aligned with the drill guide opening and template opening.
